(19) United States
(12) Patent Application Publication
(10) Pub. No.: US 2016/0192983 A1
(43) Pub. Date: Jul. 7, 2016

(54) GUIDING CATHETER HAVING A STABILIZING MECHANISM AND METHOD FOR INSERTING A GUIDING CATHETER

(71) Applicant: OLYMPUS WINTER & IBE GMBH, Hamburg (DE)

(72) Inventors: German KLINK, Berlin (DE); Andreas HÖRLLE, Berlin (DE); Carlos GROTH, Berlin (DE)

(73) Assignee: OLYMPUS WINTER & IBE GMBH, Hamburg (DE)

(21) Appl. No.: 14/912,257

(22) PCT Filed: Jun. 16, 2014
(86) PCT No.: PCT/EP2014/062537

§ 371 Date: Feb. 16, 2016

(30) Foreign Application Priority Data
Aug. 13, 2013 (DE) 10 2013 216 030.5

(51) Int. Cl. A61B 18/14; A61M 25/01

(52) U.S. Cl. CPC A61B 18/1492; A61M 25/01; A61M 25/0147; A61B 2018/00577

(57) ABSTRACT

The invention relates to a guiding catheter for inserting an applicator into body lumens in an insertion direction, including a guiding sleeve, which surrounds a lumen, a deflecting mechanism, which is arranged and designed to deflect a distal end of the guiding sleeve from the insertion direction, and a stabilizing mechanism, which is arranged and designed to support the distal end of the guiding sleeve on a wall bounding the body lumen. The invention further relates to a method for inserting a guiding catheter into a body lumen in an insertion direction.

The invention relates to a guiding catheter for inserting an applicator into body lumen along an insertion direction.

The invention further relates to an electrosurgical system with a guiding catheter and to a method for inserting a guiding catheter into a body lumen along an insertion direction.

Guiding catheters of the aforementioned type are known in the prior art and are used in particular to guide applicators therein, for example electrosurgical instruments for the coagulation and/or ablation of biological tissue and/or deposits, which applicators are arranged movably, in particular movably in an insertion direction relative to the guiding catheter, in a lumen of a guiding sleeve of the guiding catheter.

Insertion direction is understood here as a direction in which a guiding catheter is inserted into a body lumen. On account of anatomical circumstances, and since a guiding catheter can be designed to be bendable, the insertion direction is not necessarily an ideal straight line. Therefore, the feed direction in which the guiding catheter is substantially advanced is considered to be the insertion direction. Therefore, the insertion direction also substantially corresponds to a longitudinal axis of the guiding catheter.

The guiding catheter serves here for inserting an applicator as far as a target location or in front of a target location in the body lumen, in particular in a body tissue adjoining the body lumen. In bronchoscopy, or when treating bronchial carcinoma for example, this target location lies in the bronchi of the lungs. In order to reach the target location with a distal applicator tip, it is often necessary to puncture the body tissue adjoining the body lumen. Particularly if the target location lies in the bronchi, it is necessary for an applicator tip to puncture a bronchial wall, which may be cartilaginous.

It is therefore an object of the present invention to make available a guiding catheter and a method for inserting a guiding catheter, which make it easier to bring an applicator to the target location. In particular, it is an object of the present invention to make available a guiding catheter and a method for inserting a guiding catheter which allow a bronchial wall to be punctured more easily and as precisely as possible.

According to the invention, this object is achieved by a guiding catheter for inserting an applicator into body lumen along an insertion direction, comprising a guiding sleeve, which encloses a lumen, and a deflecting mechanism, which is arranged and designed to deflect a distal end of the guiding sleeve from the insertion direction, and further comprising a stabilizing mechanism, which is arranged and designed to support the distal end of the guiding sleeve on a wall delimiting the body lumen.

The invention is based on the recognition that, for bringing an applicator with pinpoint accuracy to the target location and for pinpoint puncturing of body tissue on or in front of the target location, it is necessary not only to align the distal end of the guiding catheter with respect to the target location, but also to prevent a displacement or change in location of the previously aligned distal end of the guiding catheter as a result of the puncturing forces and/or impulses that occur during the puncturing. The forces that are to be taken up by an anchor during the puncturing are directed counter to the puncturing direction and are therefore generally oriented obliquely or orthogonally with respect to the insertion direction.

To ensure that the distal end of the guiding sleeve can be brought up to or in front of a target location in a targeted manner, the guiding catheter comprises a deflecting mechanism by which the distal end of the guiding sleeve can be oriented with respect to the target location, in particular with respect to a wall delimiting the body lumen.

The distal end of the guiding catheter is preferably an open end, such that a distal end of the applicator or an applicator tip, also referred to as a probe tip, can emerge through the distal, open end of the guiding catheter. Therefore, by aligning the distal end of the guiding sleeve, it is also possible to align the applicator tip, which emerges from the distal end of the guiding sleeve, in a targeted manner with respect to the target location prior to the puncturing.

In order also to be able to ensure such pinpoint alignment during and after the puncturing, the guiding catheter according to the invention has a stabilizing mechanism. Stabilizing the distal end of the guiding sleeve on a wall delimiting the body lumen has the advantage that the distal end of the guiding sleeve (after it has been inserted near the target location and has been oriented with respect to the target location by the deflecting mechanism) can be stabilized in this position by being supported on a wall of the body lumen lying opposite the target location. In this way, an unwanted and/or uncontrolled change in position of the guiding catheter when puncturing the wall is avoided or reduced, since the forces that arise during the puncturing can preferably be taken up, at least for the most part, via the stabilizing mechanism. For this purpose, the stabilizing mechanism is preferably oriented such that the forces to be taken up can be taken up as pressure forces and/or bending moments in the stabilizing mechanism and can be introduced into the opposite wall.

Particularly when puncturing a cartilaginous bronchial wall, relatively high puncturing forces and corresponding impulses occur, which may lead to a movement and/or a change in position of the guiding catheter and of the applicator. This can have the effect that the guiding catheter originally aligned with respect to the target location is situated at a position deviating from this target position. This may have a negative impact on the treatment outcome. Stabilizing the distal end of the guiding catheter by supporting it on an opposite wall, in particular a bronchial wall, simplifies the puncturing of the opposite wall at the precise target position.

The supporting of the distal end of the guiding sleeve on a wall of the body lumen lying opposite the target location is preferably effected such that the stabilizing mechanism is arranged in a direction orthogonal to the insertion direction or at least oblique to this direction between the target location and an opposite wall of the body lumen. Since the insertion direction, as described above, is not always an ideal straight line, as a consequence of the nature of body lumens and as a result of at the at least partially optionally flexible guiding catheter, the stabilizing direction may also deviate accordingly from an ideal orthogonal orientation with respect to this insertion direction. However, it is preferable that the stabilizing direction, i.e. an orientation of the stabilizing mechanism in the stabilizing position between its distal end and its proximal end, extends between the target location and an opposite wall of the body lumen in such a way that forces arising as a result of the puncturing can be taken up by the stabilizing mechanism and these forces can be diverted at least for the most part into the opposite wall.

The deflecting mechanism can preferably be activated and deactivated from a proximal end of the guiding catheter such that, first of all, the guiding catheter can be inserted far enough along the insertion direction into the body lumen and the deflection of the distal end of the guiding sleeve by activation of the deflection mechanism takes place when the distal end is situated in the vicinity of the target location. An alignment of the distal end of the guiding sleeve by the deflection mechanism is particularly preferred in which the distal end is aligned with respect to a wall delimiting the body lumen, in particular a bronchial wall, and preferably also contacts this wall.

The deflecting mechanism can be formed, for example, by a pulling element, for example a pull wire or a pull cable, which is connected to a distal portion of the guiding sleeve at at least one connection point, preferably at a plurality of connection points, and can be activated, in particular tensioned, from the direction of a proximal end of the guiding catheter.

The applicator, also referred to as applicator probe or application probe, and guided in a guiding catheter, can preferably have one or two electrodes on its distal portion, to which electrodes a radiofrequency AC voltage can be applied. Monopolar applicators require only one electrode. During the application, this one electrode interacts with a large-area return or neutral electrode, which is likewise connected to the body of a patient. For a bipolar application, applicators are provided with at least two electrodes. Such a bipolar electrosurgical instrument preferably has an elongate applicator shaft and two coagulation or ablation electrodes, which are arranged in succession in the longitudinal direction of the applicator shaft on the applicator shaft, respectively form a surface portion of the applicator shaft and are electrically insulated from each other by an insulator. A radiofrequency (RF) voltage with different potentials (bipolar) can be applied to such coagulation or ablation instruments, as a result of which the tissue surrounding the electrodes is heated to the extent that the body's own proteins denature. Coagulation or ablation instruments can also have a mechanically cutting/puncturing tip, for example a trocar, or a cutting electrode.

In a first preferred embodiment of the guiding catheter, provision is made that the stabilizing mechanism is arranged on a distal portion of the guiding sleeve. This positioning of the stabilizing mechanism is advantageous since a distal portion specifically, i.e. a portion of the guiding sleeve adjoining the distal end of the guiding sleeve and extending from the latter in the proximal direction, can contribute in particular, by means of stabilization, to ensuring that the distal end of the guiding sleeve maintains the intended position during the puncturing. In particular since the stabilizing mechanism provides support on a wall lying opposite the target location and delimiting the body lumen, it is recommended to arrange the stabilizing mechanism on the distal portion of the guiding sleeve, since many body lumens have an extent that allows a distal portion of the guiding sleeve and a stabilizing mechanism arranged thereon to be supported on a wall lying opposite a target location.

Preferably, the stabilizing mechanism comprises at least one stabilizing element. Such a stabilizing element is preferably arranged and designed in such a way that when providing stabilization, i.e. when providing support, it is subjected in particular to pressure and/or a bending load. The stabilizing mechanism preferably further comprises a base element. The base element is preferably arranged on a proximal end of the stabilizing mechanism.

A distal end of the stabilizing mechanism is preferably connected to the distal end of the guiding sleeve. This connection can be direct or indirect. In particular, it is preferable that a distal end of the at least one stabilizing element is connected to the distal end of the guiding sleeve. A reinforcing element, for example a reinforcing ring, can for example be arranged at the distal end of the guiding sleeve and serves to connect the distal end of the guiding sleeve to a distal end of the stabilizing mechanism.

In a further embodiment, provision is also preferably made that a proximal end of the stabilizing mechanism is connected to a proximal end of a distal portion of the guiding sleeve so as to be movable along the insertion direction. This connection too can be direct or indirect. The proximal end of the stabilizing mechanism is preferably formed by the base element, and the base element is preferably connected to the proximal end of the distal portion of the guiding sleeve so as to be movable along the insertion direction. A movable arrangement of the proximal end of the stabilizing mechanism on the guiding sleeve of this kind makes it possible to change the position of the proximal end of the stabilizing mechanism relative to the guiding sleeve. In particular, it is preferable that, by moving the proximal end of the stabilizing mechanism in the insertion direction, the stabilizing mechanism is brought from a release position to a stabilizing position and, by a movement of the proximal end counter to the insertion direction, the stabilizing mechanism can be brought back again from a stabilizing position to the release position.

The stabilizing mechanism is preferably arranged substantially outside the lumen of the guiding sleeve. In this way, the stabilizing mechanism, in particular between its distal end and its proximal end, can adopt a distance from the guiding sleeve, which can facilitate the support.

A particularly preferred embodiment is one in which the stabilizing mechanism has, between its distal end and its proximal end, an extent that is greater than three times a diameter of the guiding catheter, preferably greater than five times the guiding catheter, preferably greater than ten times the guiding catheter.

The guiding catheter preferably has a diameter (external diameter) which is smaller than the body lumen in which or adjacent to which the target location is situated, in order to permit the insertion of the guiding catheter into the body lumen as far as the target location. To ensure that the stabilizing mechanism can support itself on a wall of the body lumen lying opposite this target location, it is preferable that the stabilizing mechanism, at least in its stabilizing position, has an extent between its distal end and its proximal end that is sufficient to allow the stabilizing mechanism to contact the wall lying opposite the target location, so as to be able to introduce forces there. Particularly if a distal end of the stabilizing mechanism is arranged at the distal end of the guiding sleeve, it is preferable that, at least in the stabilizing position, the proximal end of the stabilizing mechanism can reach an opposite wall. For this purpose, the stabilizing mechanism preferably has a length or extent which is many times, in particular at least three times, preferably at least five times and particularly preferably at least ten times, the diameter of the guiding catheter.

Moreover, an embodiment is preferred in which the at least one stabilizing element has a greater deformation resistance than a distal portion of the guiding sleeve. In particular, it is preferable that the at least one stabilizing element has a higher modulus of elasticity than a distal portion of the guiding sleeve. The deformation resistance and/or the modulus of elasticity are preferably compared here at ambient temperature. A comparison is preferred in particular at the use temperature, preferably at body temperature.

The guiding catheter preferably comprises an activation mechanism which is designed to move the stabilizing mechanism from a stabilizing position to a release position and vice versa. This means that the stabilizing mechanism can be activated via the activation mechanism. In particular, the base element can preferably be moved in or counter to the insertion direction by the activation mechanism.

In a further advantageous embodiment of the guiding catheter, provision is made that the activation mechanism is arranged at a proximal end of the guiding catheter. For example, the activation mechanism can be arranged on a proximal handle of the guiding catheter or can be integrated therein. For example, the activation mechanism can be designed as a lever on a proximal handle of the guiding catheter.

Preferably, the activation mechanism is connected to the stabilizing mechanism directly or indirectly. Such a use can be permitted by different mechanisms.

Preferably, the activation mechanism is connected to the stabilizing mechanism via at least one activation element, preferably via two, three or more activation elements. The one or more activation elements are preferably designed in particular to transmit tensile forces and/or pressure forces and/or bending moments. In particular, such an activation element is preferably connected to the base element of the stabilizing mechanism in such a way that, by an activation of the activation element, the base element can be moved relative to the guiding sleeve in or counter to the insertion direction.

In another preferred embodiment of the guiding catheter, provision is made that the stabilizing mechanism can be fixed, preferably releasably, in the stabilizing position. In particular, a locking mechanism can be provided which is designed to fix the stabilizing mechanism, preferably releasably, in the stabilizing position. This preferably releasable fixing of the stabilizing mechanism in the stabilizing position, for example via a locking mechanism, has the advantage that the stabilizing mechanism is held in the stabilizing position until it is actively released again from the stabilizing position. In this way, unwanted release of the stabilizing position can be prevented. Appropriate securing or fixing of the stabilizing mechanism in the stabilizing position is advantageous particularly during puncturing, when high puncturing forces and/or impulses occur.

In a preferred embodiment variant, provision is made that the stabilizing mechanism has two, three or more stabilizing elements. The stabilizing elements are preferably arranged in succession in the insertion direction, in particular in the release position. The stabilizing elements and/or the base element can have a rod-shaped or strip-shaped design, for example. It is moreover preferable that the base element is designed as a feed wire.

In particular, it is preferable that two adjacent stabilizing elements are connected to each other by a joint. Therefore, in a design with two stabilizing elements, these two stabilizing elements are preferably connected to each other by a joint; in the case of three or more stabilizing elements, two stabilizing elements arranged adjacent to each other are preferably connected to each other by a joint.

It is moreover preferable that the base element is connected to a proximal one of the stabilizing elements via a joint. Proximal stabilizing element denotes the stabilizing element which is farthest away from the distal end of the guiding catheter.

The joint via which two adjacent stabilizing elements are connected to each other is preferably designed as a joint that closes on one side. In particular, it is preferable that the joint is arranged and designed in such a way that the two stabilizing elements connected via the joint can be arranged in three orientations, namely both in the insertion direction, both orthogonal or oblique with respect to the insertion direction, or an orientation in which both stabilizing elements enclose an angle of 30 to 120°, preferably of approximately 90°, wherein the distal one of the two stabilizing elements is oriented orthogonally or obliquely with respect to the insertion direction and the proximal one of the two stabilizing elements is oriented in the insertion direction. The change between the orientations preferably takes place by pressing and/or pulling being effected in and/or counter to the insertion direction via the base element.

If several joints are provided, it is preferable that two or more joints, preferably all of the joints, are designed as joints that close on one side.

In an alternative embodiment, provision is made that the stabilizing mechanism has precisely one stabilizing element. It is moreover preferable that the base element extends at least over a part of the circumference of the guiding sleeve. It is moreover preferable that the base element has an annular shape and/or the stabilizing element has a strip-shaped design.

The at least one stabilizing element extends with its main direction of extent preferably between the distal end of the guiding sleeve and the base element. Moreover, the at least one stabilizing element is preferably designed as plate.

In this embodiment, the stabilizing mechanism is preferably formed by the deflecting mechanism, i.e. the stabilizing mechanism is activated at the same time as the distal end of the guiding sleeve is deflected. The stabilizing element, of which the proximal end is moved along the guiding sleeve in the insertion direction by the base element, thus causes a deflection of the distal end of the guiding sleeve and at the same time provides support on a wall lying opposite the target location. This takes place in particular by a bending deformation of the stabilizing element, which bending deformation is generated by the movement of the base element in the direction of the distal end of the guiding sleeve.

It is moreover preferable that the base element and/or the stabilizing element has at least one locking hook which is arranged and designed to interact with at least one complementary locking element during a movement of the base element relative to the guiding sleeve counter to the insertion direction. The at least one locking hook and the at least one complementary locking element are preferably arranged and designed in such a way that the locked connection can be released again by application of a certain minimum tensile force. Preferably, several locking hooks of this configuration are present that interact with the at least one or more complementary locking elements. The at least one locking element can be formed on a proximal end, for example a handle, of the guiding catheter or of an endoscope, on the guiding sleeve, on the base element and/or on a distal outlet of a working channel of an endoscope that receives the guiding catheter.

According to a further aspect of the invention, the aforementioned object is achieved by an electrosurgical system with an above-described guiding catheter and with an applicator which is designed as an electro-surgical instrument and which is guided movably in the guiding sleeve of the guiding catheter.

According to a further aspect of the present invention, the aforementioned object is achieved by a method for inserting a guiding catheter into a body lumen along an insertion direction, said method comprising the steps of: inserting a distal end of a guiding sleeve into a body lumen along the insertion direction, deflecting the distal end of the guiding sleeve from the insertion direction, and supporting the distal end of the guiding sleeve on a wall delimiting the body lumen.

The method is suitable in particular for inserting an above-described guiding catheter or an above-described electrosurgical system up to or in front of a target location in a body tissue that adjoins a body lumen.

The electrosurgical system and the developments thereof and the method and the developments thereof preferably have features or method steps which make them particularly suitable for being used with a guiding catheter according to the invention and with the developments thereof.

Regarding the advantages, embodiment variants and embodiment details of the electrosurgical system and the developments thereof, and of the method and the developments thereof, reference is made to the above description of the corresponding features of the guiding catheter.

A preferred embodiment of the invention is described by way of example on the basis of the attached figures, in which:

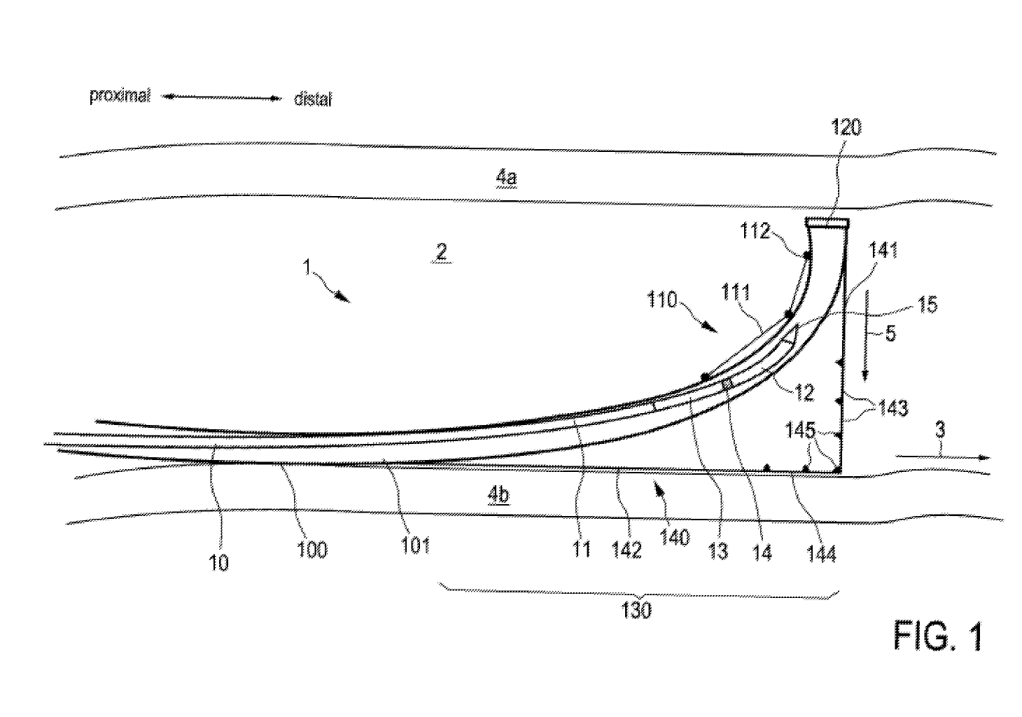

FIG. 1 shows a first illustrative embodiment of a guiding catheter according to the invention;

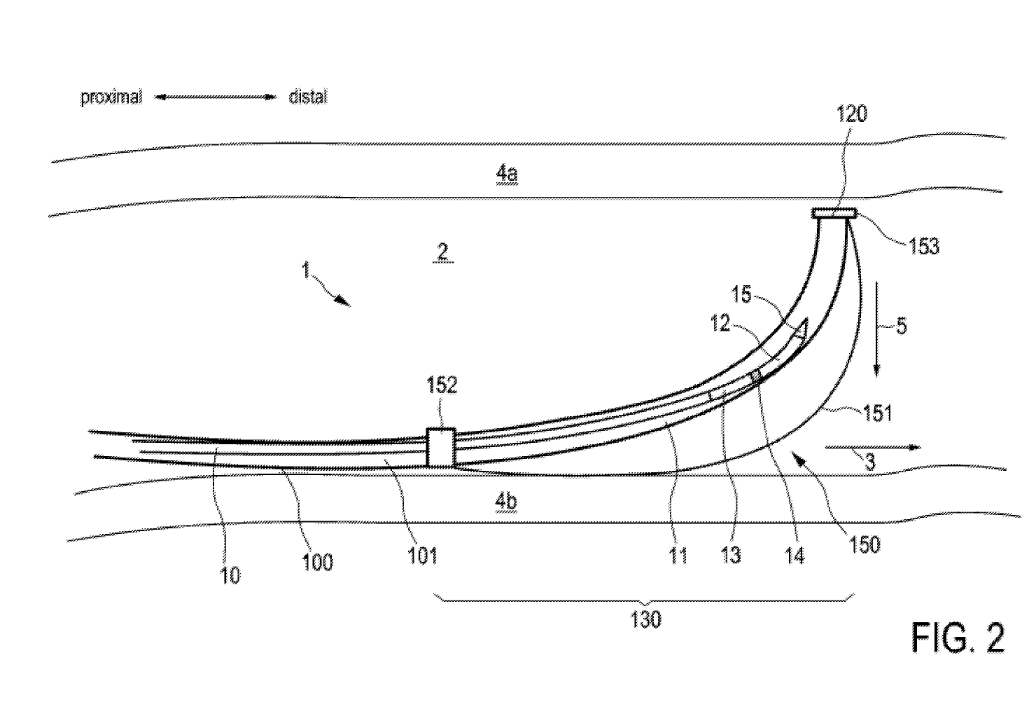

FIG. 2 shows a second illustrative embodiment of a guiding catheter according to the invention; and

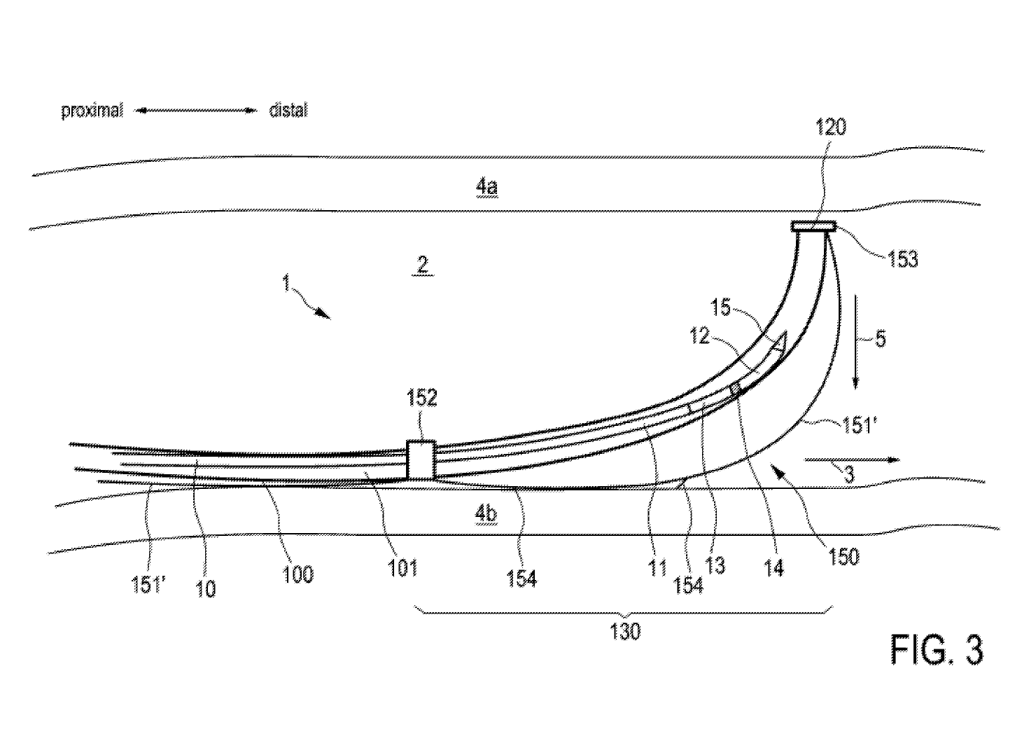

FIG. 3 shows the guiding catheter according to FIG. 2 with locking hooks on the stabilizing element; and

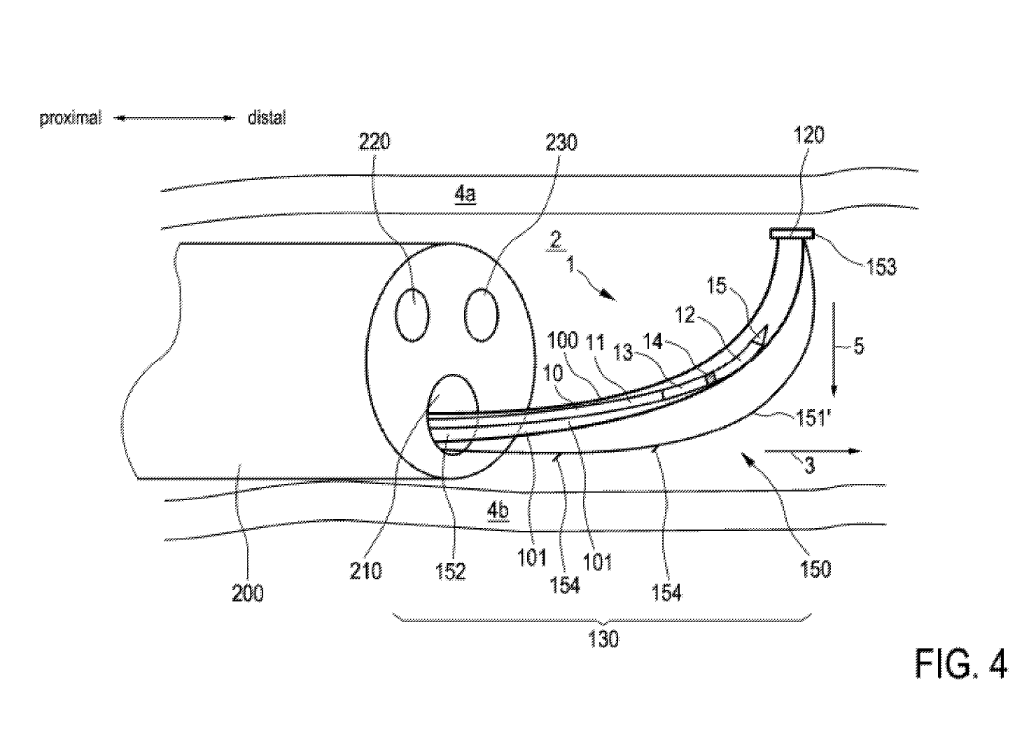

FIG. 4 shows the guiding catheter according to FIG. 3 with the complementary locking element formed at the outlet of the working channel of an endoscope.

FIGS. 1 to 4 depict illustrative embodiments of a guiding catheter according to the invention. Elements which are equivalent or substantially functionally equivalent are provided with the same reference signs.

Unless stated otherwise, the following description relates to both embodiments.

FIGS. 1 to 4 depict a guiding catheter 1 for inserting an applicator 10 into a body lumen 2 along an insertion direction 3. The guiding catheter 1 comprises a guiding sleeve 100, which encloses a lumen 101. FIG. 4 moreover shows an endoscope 200 with a working channel 120, which receives the guiding catheter 1, and with an optical channel 220 and a flushing channel 230.

An applicator 10 is arranged movably, in particular movably relative to the guiding catheter in insertion direction 3, in the lumen 101 of the guiding catheter 1. The applicator 10 is formed as a bipolar electro-surgical instrument with an elongate applicator shaft 11 and two coagulation or ablation electrodes 12, 13, which are arranged on the applicator shaft 11, in succession in the longitudinal direction of the applicator shaft, and which each form a surface portion of the applicator shaft 11. The distal electrode 12 and the proximal electrode 13 are electrically insulated from each other by an insulator 14. The insulator 14 is arranged coaxially with respect to the electrodes 12, 13 and likewise forms a surface portion of the applicator shaft 11. An applicator tip 15 is formed on the distal coagulation or ablation electrode 12. By way of example, a mechanically cutting and/or puncturing tip, for example a trocar, can be arranged on the distal applicator tip 15. A cutting electrode can also be provided on the applicator tip 15. Overall, with the exception of the applicator tip 15, a cylindrical design with a substantially constant circular cross section is provided for the applicator shaft 11.

The distal end 120 of the guiding sleeve 100 preferably has an open configuration, such that a distal end of the applicator 10 with an applicator tip 15 can emerge from the distal end 120 of the guiding sleeve 100 in order to be able to advance to a target location in the tissue.

The applicator 10, in particular the applicator shaft 11, is preferably flexible in order to be able to follow a deflection of a distal portion 130 of the guiding sleeve 100 by the deflecting mechanism 110.

The target location, at which a treatment is intended to be performed by means of the applicator 10, is preferably situated in the region behind the contact point of the distal end 120 of the guiding sleeve 100 on the bronchial wall 4a.

The wall, preferably a bronchial wall, is punctured by means of the applicator tip 15 in order to allow the applicator 10 to penetrate into the wall 4a and thus advance to the target location. High puncturing forces and impulses may occur particularly when puncturing a bronchial wall, and these may lead to a displacement or change in position of the distal end 120 of the guiding catheter 1.

A stabilizing mechanism is provided in order to prevent this. The stabilizing mechanism serves to support the distal end 120 of the guiding sleeve 100 on the opposite wall 4b. FIGS. 1 and 2 depict two different embodiments for such a stabilizing mechanism that are described below. FIG. 1 shows a stabilizing mechanism 140 and a deflecting mechanism 110, while FIG. 2 shows a stabilizing mechanism 150 which at the same time is also a deflecting mechanism.

In the embodiment variant shown in FIG. 1, the guiding catheter 1 moreover has a deflecting mechanism 110 by means of which the distal end 120 of the guiding sleeve can be deflected from the insertion direction 3. For this purpose, in the illustrative embodiment shown in FIG. 1, three connection points 112 are provided on a distal portion 130 of the guiding sleeve 100, at which connection points 112 a pulling element designed as a pull wire 111 is connected to the distal portion 130 of the guiding sleeve 100. The deflecting mechanism 110 can preferably be tensioned from the direction of a proximal end of the guiding catheter. For this purpose, the pull wire 111 can be routed, for example, as far as the proximal end of the guiding catheter (inside or outside the guiding sleeve 100). By applying tension to the pull wire 111, the distal end 120 is deflected, as shown in FIG. 1, and oriented in the direction of a wall 4a and preferably brought into contact with this wall 4a.

FIG. 1 shows an embodiment variant of a guiding catheter 1 with a stabilizing mechanism 140 which has several stabilizing elements 141, 143, 144 and a base element 142. The distal stabilizing element 141 is longer than the other stabilizing elements 141, 143, 144 and, with its distal end, is connected to the distal end 120 of the guiding sleeve 100. For this purpose, the distal end 120 of the guiding sleeve 100 can have a reinforcing ring. The stabilizing elements 141, 143, 144 and the base element 142 are arranged in succession in insertion direction 3, at least in the release position (not shown). In the stabilizing position shown in FIG. 1, the stabilizing elements 141, 143, 144 and the base element 142 are arranged in such a way that they form a 90° angle, wherein the distal stabilizing element 141 is orthogonal to the insertion direction 3, and the proximal stabilizing element 140 and the base element 142 are oriented in insertion direction 3. The other stabilizing elements 413 are arranged in part in insertion direction 3 and in part orthogonal to the insertion direction 3.

Two adjacent stabilizing elements 141, 143, 144 are in each case connected by a joint 145 that closes on one side. As can be seen in FIG. 1, the joints 145 can adopt different positions, wherein the respectively adjacent stabilizing elements are either oriented in the same direction or the two stabilizing elements connected by a joint enclose an angle, for example an angle of 90° as shown here. By advancing the base element 142 in insertion direction 3 or pulling the base element 142 back counter to the insertion direction 3 (preferably by means of an activation mechanism not shown), the joints 145 closing on one side can be brought to their different orientations. In the release position, the base element 142 and all of the stabilizing elements 141, 143, 144 are preferably arranged in succession in insertion direction 3. In the stabilizing position, at least one of the joints that close on one side is blocked in a position in which the stabilizing elements arranged adjacent thereto enclose an angle of less than 180°, in particular an angle of 30° to 120°, in particular an angle of 90°.

In this stabilizing position shown in FIG. 1, the forces that arise during puncturing can then be taken up in direction 5 via the stabilizing mechanism 140 and can be introduced at the wall 4b lying opposite the target location.

FIGS. 2 to 4 show an alternative embodiment variant of a guiding catheter 1 with an alternative stabilizing mechanism 150. The stabilizing mechanism 150 in FIGS. 2 to 4 has precisely one stabilizing element 151, 151′. In FIGS. 2 and 3, the stabilizing element 151, 151′ is connected to an annular base element 152. The annular base element 152, which can also be designated as a guide ring, extends over the circumference of the guiding sleeve 100. Although no base element is shown in FIG. 4, such a base element may also be present in the embodiment according to FIG. 4 (and would then be arranged inside the working channel 210 of the endoscope 200 and would not be visible). In principle, it is also possible for several base elements to be provided, preferably distributed along the length of the guiding catheter 1.

In the variant shown in FIG. 2, the base element 152 has, on its inner circumference, several locking hooks (not shown) with which the base element 152 can be fixed in the stabilizing position shown in FIG. 2. For this purpose, the locking hooks work with complementary locking elements, for example ribs (not shown), of the guiding sleeve 100 after the base element 152 has been moved in insertion direction 3 and pulled back slightly counter to the insertion direction 3. The movement or activation of the base element 152 preferably takes place via an activation mechanism (not shown), which can preferably be actuated from the direction of the proximal end of the guiding catheter 1. In FIGS. 3 and 4, several locking hooks 154 are arranged on the stabilizing element 151′ and interact with at least one complementary locking element. In the variant shown in FIG. 3, the locking element is formed by the base element 152. If several base elements are provided, the locking element is preferably formed by the distal one of the base elements. In the variant shown in FIG. 4, the locking element is formed by the distal outlet of a working channel 210 of an endoscope 200 which receives the guiding catheter.

The stabilizing element 151, 151′ is formed as plate, preferably as flexible sheet-metal plate, and is connected by its distal end 153 to the distal end 120 of the guiding sleeve 100. For this purpose, the distal end 120 of the guiding sleeve 100 can have a reinforcing ring.

As can be seen in FIGS. 2 to 4, the stabilizing mechanism 150 acts at the same time as a deflecting mechanism since, during the advance of the base element 152 in insertion direction 3, a bending deformation of the plate 151, 151′ occurs which leads to a corresponding deflection of the distal end 120 of the guiding sleeve 100. The bending of the stabilizing element 151, 151′, brought about by the advance of the base element 152 in insertion direction 3, thus causes the deflection or orientation of the distal end of the guiding sleeve 100 in the direction of the target location and, at the same time, the stabilizing element 150 that is deformed by bending ensures that forces arising in direction 5 during puncturing can be introduced by way of the plate into the wall 4b lying opposite the target location.

For this purpose, an extent of the stabilizing element 151, 151′ between its distal end 153 and its proximal end connected to the base element 152 is greater than three times the diameter of the guiding catheter 1 and therefore, as can be seen, also greater than the diameter of the body lumen 2 between the two walls 4a and 4b. It is ensured in this way that, even in the event of an oblique or curved orientation of the stabilizing element 151, 151′ in the stabilizing position shown in FIGS. 2 to 4, the stabilizing element 151, 151′ is arranged on the distal end 120 of the guiding sleeve 100 and is oriented together with this distal end on or in front of the wall 4a in the area of the target location and at the same time can be supported on the opposite wall 4b.

In all of the variants shown in FIGS. 1 to 4, the stabilizing mechanism 140, 150 can preferably be released again from the stabilizing position after the puncturing has taken place, either directly after said puncturing or preferably after treatment is carried out. For this purpose, the base element 142, 152 is preferably pulled counter to the insertion direction 3. This pull can preferably be applied via the activation mechanism.

In the variant shown in FIG. 1, this pull preferably has the effect that the joints 145 closing on one side bring the adjacent stabilizing elements 141, 413, 144 back to the release position. In the variants shown in FIGS. 2 to 4, this pull is preferably stronger than a resistance force of the locked connection, such that the locked connection is released when a minimum pulling force is exceeded.

### LIST OF REFERENCE SIGNS

1 guiding catheter
2 body lumen
3 insertion direction
4a wall delimiting the body lumen
4b wall delimiting the body lumen
5 direction of the forces that are to be supported during puncturing
10 applicator
11 applicator shaft
12 distal coagulation or ablation electrode
13 proximal coagulation or ablation electrode
14 insulator
15 applicator tip
100 guiding sleeve
101 lumen of the guiding sleeve
110 deflecting mechanism
111 pulling element designed as a pull wire
112 connection points
120 distal end of the guiding sleeve
130 distal portion of the guiding sleeve
140 stabilizing mechanism
141 distal stabilizing element
142 base element
143 stabilizing element
144 proximal stabilizing element
145 joint closing on one side
150 deflecting and stabilizing mechanism
151 stabilizing element
151′ stabilizing element
152 base element
153 distal end of the stabilizing element
154 locking hook
200 endoscope
210 working channel
220 optical channel
230 flushing channel

1. A guiding catheter for inserting an applicator into body lumen along an insertion direction comprising
a guiding sleeve which encloses a lumen,
a deflecting mechanism which is arranged and designed to deflect a distal end of the guiding sleeve from the insertion direction,
further comprising a stabilizing mechanism which is arranged and designed to support the distal end of the guiding sleeve on a wall delimiting the body lumen.

2. The guiding catheter according to claim 1, wherein the stabilizing mechanism comprises at least one stabilizing element.

3. The guiding catheter as claimed in claim 1, wherein the stabilizing mechanism comprises a base element.

4. The guiding catheter as claimed in claim 1, wherein a proximal end of the stabilizing mechanism is connected to a proximal end of a distal portion of the guiding sleeve so as to be movable along the insertion direction.

5. The guiding catheter as claimed in claim 1, wherein the stabilizing mechanism has, between its distal end and its proximal end, an extent that is greater than three times a diameter of the guiding catheter.

6. The guiding catheter as claimed in claim 1, wherein the stabilizing mechanism can be fixed in the stabilizing position.

7. The guiding catheter as claimed in claim 1, wherein the stabilizing mechanism has two, three or more stabilizing elements.

8. The guiding catheter as claimed in claim 1, wherein two adjacent stabilizing elements are connected to each other by a joint.

9. The guiding catheter as claimed in the claim 8, wherein the joint is designed as a joint that closes on one side.

10. The guiding catheter as claimed in claim 1, wherein the stabilizing mechanism has precisely one stabilizing element.

11. The guiding catheter as claimed in claim 1, wherein the base element extends at least over a part of the circumference of the guiding sleeve.

12. The guiding catheter as claimed in claim 1, wherein the stabilizing mechanism is formed by the deflecting mechanism.

13. The guiding catheter as claimed in claim 1, wherein the base element and/or the stabilizing element has at least one locking hook which is arranged and designed to interact with at least one complementary locking element during a movement of the base element relative to the guiding sleeve counter to the insertion direction.

14. An electrosurgical system with a guiding catheter as claimed in claim 1 and with an applicator which is designed as an electrosurgical instrument and which is guided movably in the guiding sleeve of the guiding catheter.

15. A method for inserting a guiding catheter into a body lumen along an insertion direction, said method comprising the steps of:
inserting a distal end of a guiding sleeve into a body lumen along the insertion direction,
deflecting the distal end of the guiding sleeve from the insertion direction,
supporting the distal end of the guiding sleeve on a wall delimiting the body lumen.
